(19) United States
(12) Patent Application Publication
(10) Pub. No.: US 2014/0303442 A1
(43) Pub. Date: Oct. 9, 2014

(54) LAPAROSCOPIC DEVICE

(71) Applicant: Daniel Sherwin, Jerusalem (IL)

(72) Inventor: Daniel Sherwin, Jerusalem (IL)

(73) Assignee: CLEANOSCOPE INC., Antwerpen (BE)

(21) Appl. No.: 14/343,372

(22) PCT Filed: Sep. 10, 2012
(86) PCT No.: PCT/IL2012/000337

§ 371 Date: Jun. 10, 2014

(30) Foreign Application Priority Data
Sep. 12, 2011 (IL) 215106

(51) Int. Cl. A61B 1/06; A61B 1/07; A61B 1/313

(52) U.S. Cl. CPC A61B 1/0676; A61B 1/313; A61B 1/07; A61B 1/0684
USPC 600/178

(57) ABSTRACT

The invention is a laparoscopic device comprising an internal light source located at the distal end of the handle section, a central core comprising an optical system, and a light guide in the form of a cylindrical tube that is slipped over the central core. The light guide is adapted to cause the light emitted by the internal light source to propagate through it by internal reflection until light exits the light guide at the distal end of the laparoscope.

### FIELD OF THE INVENTION

The invention is in the field of medical instrumentation. Specifically the invention is from the field of laparoscopic devices.

### BACKGROUND OF THE INVENTION

Endoscopes and laparoscopes are devices used in medical applications for observation, diagnosis, and treatment, e.g. biopsy or cauterization inside a human or animal body and in industrial applications for inspection in hard to reach locations, e.g. to inspect the turbine blades in jet engines. They comprise an objective lens system, normally at the distal end and means for carrying the images gathered to the proximal end of the device where they can be directly viewed or displayed allowing the operator to observe the space in front of the objective lens. The objective lens can be part of a camera, an optical relay system, or can focus the image onto a fiber optic cable that transfers the image to the viewer.

In conventional laparoscopic devices the light necessary to illuminate the region in front of the objective lens is transmitted from the proximal to the distal end of the insertion tube by optical fibers. It is not uncommon for the fragile optical fibers to break rendering the instrument unusable unless costly repairs are undertaken.

Another problem associated with conventional laparoscopic devices is the expense and “clown time” involved in sterilization procedures that must be carried out between procedures. An associated problem is “wear and tear”, especially on the optical and electronic components, that is caused by the high temperatures of autoclaving or harsh chemicals and shortens the useful lifetime of the device.

It is a purpose of the present invention to provide laparoscopic devices that overcome these drawbacks of the conventional devices.

Further purposes and advantages of this invention will appear as the description proceeds.

### SUMMARY OF THE INVENTION

The invention is a laparoscope comprising a handle section, a central core comprising an optical system having an objective lens at the distal end of the central core, and an internal light source.

The laparoscope of the invention is characterized in that the internal light source is located at the distal end of the handle section and the laparoscope comprises a light guide in the form of a cylindrical tube that is slipped over the central core. The light guide is adapted to cause the light emitted by the internal light source to propagate through it by internal reflection until light exits the light guide at the distal end of the laparoscope. The light guide can be made from either glass or plastic.

In embodiments of the laparoscope of the invention the proximal end of the light guide is shaped like a lens to focus the light propagated through it at a predetermined distance in front of the objective lens or to control the size of the volume illuminated by the light or to control the angular position of the center of the illuminated volume relative to the longitudinal symmetry axis of the central core of the laparoscope.

Embodiments of the laparoscope comprise a metal or elastomer outer protector tube that is slipped over the light guide.

Embodiments of the laparoscope of the invention comprise a collar which is adapted to fit over the light guide and to be attached to the distal end of the handle section in order to keep the light guide firmly attached to the handle section.

Embodiments of the laparoscope of the invention comprise a transparent window that is sealed at the distal end of either the light guide or the protector tube.

In embodiments of the laparoscope of the invention the internal light source is either LEDs or optical fibers. In embodiments of the laparoscope of the invention the internal light source is a ring-shaped optical fiber.

In embodiments of the laparoscope of the invention at least one of the following components are disposable: the light guide, the protector tube, and the collar.

Embodiments of the laparoscope of the invention may comprise a centrifugal lens protector unit. The centrifugal lens protector unit may be disposable.

Embodiments of the laparoscope of the invention may comprise one or more working channels.

All the above and other characteristics and advantages of the invention will be further understood through the following illustrative and non-limitative description of embodiments thereof, with reference to the appended drawings.

### BRIEF DESCRIPTION OF THE DRAWINGS

FIG. 1A is an isometric view schematically showing features of a first embodiment of the laparoscope of the invention;

FIG. 1B is a schematic cross-sectional view of the embodiment of the invention shown in FIG. 1A;

FIG. 2A is an isometric view schematically showing a second embodiment of the laparoscope of the invention;

FIG. 2B is a schematic cross-sectional view of the embodiment of the invention shown in FIG. 2A; and

FIG. 3 is a cross-sectional view of a prior art centrifugal lens protector comprised of a motor assembly and rotating disc.

### DETAILED DESCRIPTION OF EMBODIMENTS OF THE INVENTION

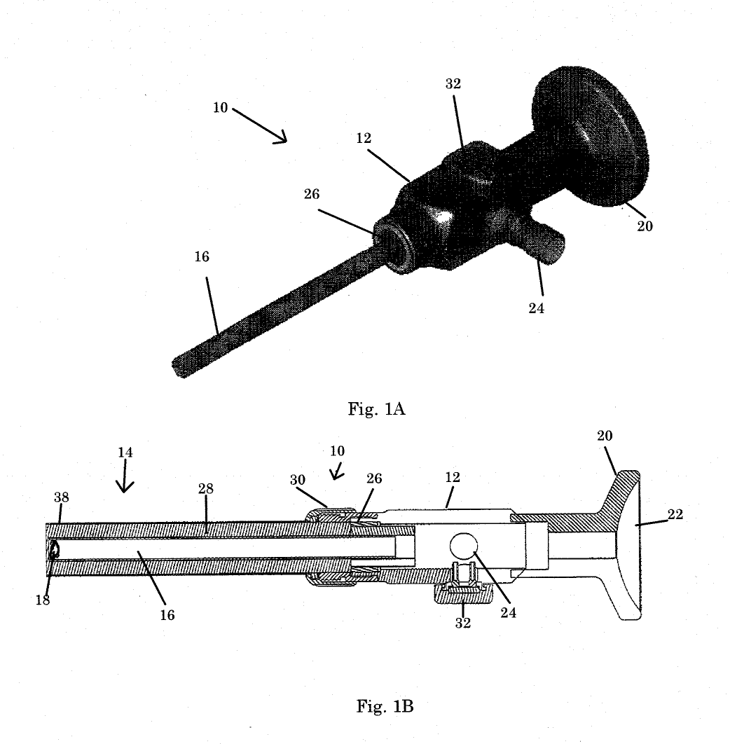

A first embodiment of the laparoscopic device 10 of the invention is shown schematically in isometric view in FIG. 1A and in a cross-sectional view in FIG. 1B. Laparoscope 10 is comprised of a handle section 12 and an insertion section 14 comprising a long central core 16. Central core 16 comprises an optical system having an objective lens 18 at the distal end of central core 12. The exact nature of the optical system, which can be any type of system known in the art, e.g. a video camera or an optical fiber bundle or optical relay system to transfer light gathered by objective lens 18 to ocular lens 22 in eyepiece 20 or a proximal image forming system and a connection to a display device (not shown in the figures), is not relevant to the present invention and therefore not discussed in detail herein.

In laparoscope 10, the internal light source 26 is not located at the distal end of insertion section 14 but at the distal end of the handle section 12. The internal light source 26 can either be, for example, LEDs that receive their electrical power through connector 32 on handle section 12 or optical fibers that carry light from an external source via connector 24. In the embodiment shown in the figures, the internal light source 26 is a ring-shaped optical fiber.

In order to convey the light emitted by internal light source 26 to the distal end of the laparoscope, where it can illuminate the region in front of objective lens 18, a transparent light guide 28 is slipped over central core 16. Light guide 28 is a cylindrical tube made of glass or plastic. The proximal end of the light guide is optically coupled to the internal light source 26. The material of which light guide 28 is made is selected such that it has an index of refraction that causes light to propagate through the guide by internal reflection until it exits the light guide at the circumference of the objective lens.

In embodiments of the invention, the proximal end of light guide 28 can be shaped like a lens to focus the light that has propagated through it at a predetermined distance in front of objective lens 18 or to control the size of the illuminated volume or the angular position of the center of the illuminated volume relative to the longitudinal symmetry axis of the central core 16 of laparoscope 10.

A metal or elastomer outer protector tube 38 can be slipped over the light guide to prevent leakage of light through the sides of the light guide and also to lend rigidity to the insertion section 14, if desired. A collar 30 fits over the light guide 28 and is screwed onto threads at the distal end of the handle section 12 in order to keep the light guide firmly attached to the handle section.

The collar 30 is fitted with seals, e.g. o-rings or other means known in the art, to prevent passage of air or liquids. A transparent window can be sealed at the distal end of either the light guide 28 or protector tube 38 to protect the objective lens 18. The combination of seals at collar 30 and a window at the distal end hermetically isolates the hollow inside of light guide 28, which is occupied by central core 16, and the handle section 12, which comprises the internal light source 26, from the exterior of the light guide.

After a procedure has been performed with laparoscope 10, collar 30 is loosened, and light guide 28 is separated from the handle portion 12. Collar 30, light guide 28, and protector tube 38, which are the only components of laparoscope 10 that have been in contact with bodily fluids or other contaminants can be discarded.

To ready endoscope 10 for another procedure it is only necessary to wipe down central core 16 and handle section 12 with a cloth that has been wet with a disinfection solution and to attach a new sterile light guide 28 and protector tube 38 by means of a new collar 30.

In embodiments of the invention at least one of light guide 28, protector tube 38, and collar 30 can be reused after being sterilized.

A problem commonly encountered when using a laparoscope is that the lens or window protecting the lens becomes at least partially occluded by body fluids and particles. In many cases the occlusion becomes so great that the laparoscope becomes inoperative. In such circumstance the lens must be cleaned. One method of cleaning the lens that has been developed is to spray the outer surface of the lens with a jet of water, air or other gas. The water or gas is supplied to one or more nozzles on the distal end of the laparoscope adjacent to the lens through channels that are an integral part of the instrument. In some instances the laparoscope must be withdrawn from the body for cleaning and reinserted, which is a time-consuming and risk-increasing procedure.

Another solution to this problem is a centrifugal lens protector. A centrifugal lens protector comprises a spinning glass disc that is placed in front of the lens, so that the debris is intercepted by the disc. The centrifugal force generated by the disc as it rotates throws the debris towards the circumference of the disc, where it falls off or sometimes accumulates but in any case no longer interferes with the image.

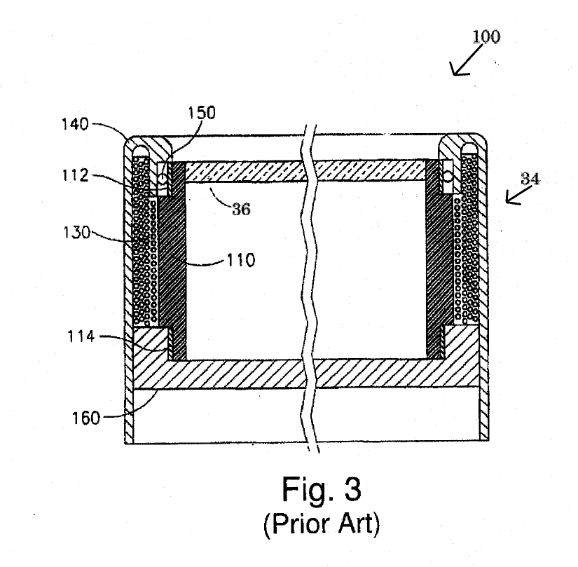

FIG. 3 is a cross-sectional view of a prior art centrifugal lens protector unit 100 that is described in U.S. Pat. No. 7,104,657 to the inventor of the present invention. The centrifugal lens protector is comprised of motor assembly 34 and rotating disc 36.

Disc 36 is provided with a plurality of axially aligned magnets 110. One end of each magnet is affixed to disc 36, for example using an adhesive or using a tension ring surrounding the magnets. Optionally, the magnets are mounted directly on disc 36, for example, being held in depressions formed in disc 102. Magnets 110 extend axially from disc 102 into the interior volume of housing 140. Disc 36 is optionally held in place by a plurality of ball or cylinder bearings 150, which enable rotation. In some embodiments the diameter of a bearing 150 is 0.2 millimeter. Alternatively or additionally, a slip-ring bearing is used. At the base of motor assembly 34, a second bearing 160 is optionally provided, for example, a slip-ring bearing.

In an exemplary embodiment, magnets 110 are interconnected by a pair of optional sliding rings 112 and 114 which serve, as tension rings, to prevent the centrifugal force of rotation from detaching the magnets from disc 36 or causing undue distortion. Sliding rings 112 and 114 slide in contact with bearings 160 and 150. In some embodiments, the life time of the lens protector unit 100 is relatively short, so wear of the bearings is not crucial. Such slip rings may be mounted on the moving part (e.g. the disc) or on the static part (e.g., housing 140).

A plurality of coils 130 are provided within the body of housing 140, at or close to the inner surface of housing 140. In some embodiments, coils 130 are fabricated by a process of depositing electroconductive material, e.g. copper or silver or platinum or an electroconductive plastic in a specially formed cavity in housing 140. Many different lithographic methods may be used for fabricating coils 130. Alternatively, the coils are deposited on a rigid or flexible substrate and the substrate is inserted into housing 140. Alternatively or additionally, coils 130 are wound on cores, optionally ferromagnetic cores. Coils 130, on their cores, are inserted into a receptive depression in housing 140.

Additional description of different embodiments of the motor assembly of the lens protector 100, including typical dimensions speeds of rotation, arrangements of the coils, etc. are given in U.S. Pat. No. 7,104,657.

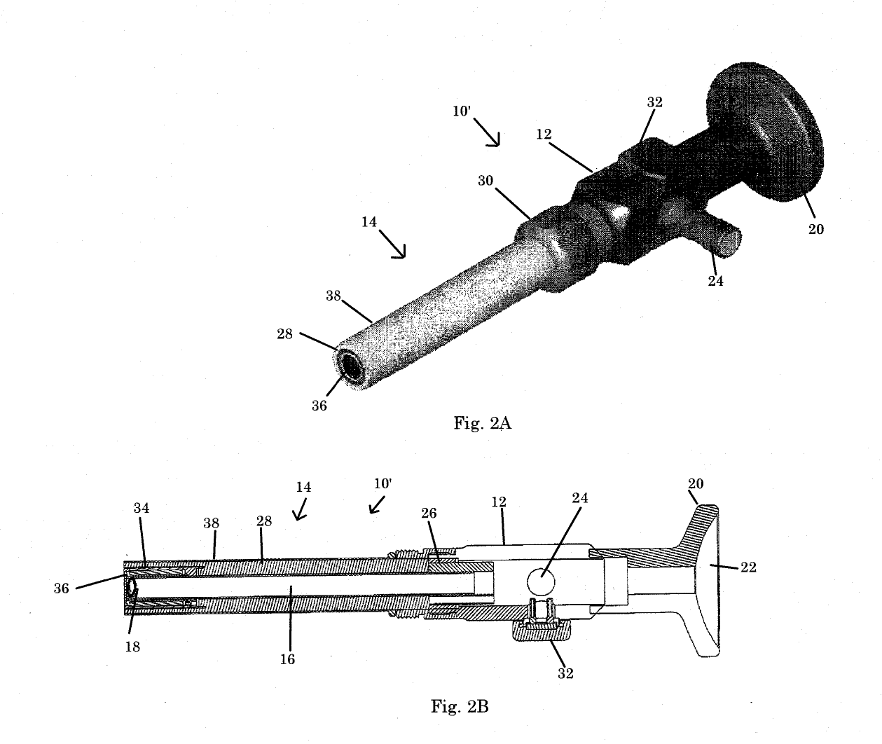

A second embodiment of the laparoscopic device 10′ of the invention is shown schematically in isometric view in FIG. 2A and in a cross-sectional view in FIG. 2B. This embodiment is identical in all respects to the first embodiment with the exception of a recess that is created at the distal end of laparoscope 10′. The hollow center of a motor assembly 34 of a centrifugal lens protector unit is slipped over the distal end of central core 16 such that the motor assembly 31 is inserted between and coaxial with the light guide 28 and central core 16 with the rotating disc 36 located proximally to objective lens 18. Connectors 32 for the electricity supply to motor assembly 34 are located on the side of the handle section 20.

In embodiments of the laparoscopes of the invention the centrifugal lens protector unit is used for only one procedure and then discarded with the other disposable components of the laparoscope.

Embodiments of the laparoscope of the invention, either with or without the centrifugal lens protector unit can be supplied with one or more working channels.

Although embodiments of the invention have been described by way of illustration, it will be understood that the invention may be carried out with many variations, modifications, and adaptations, without exceeding the scope of the claims.

1. A laparoscope comprising
a. a handle section;
b. a central core comprising an optical system having an objective lens at the distal end of said central core; and
c. an internal light source;
characterized in that said internal light source is located at the distal end of said handle section and said laparoscope comprises a light guide in the form of a cylindrical tube that is slipped over said central core;
said light guide adapted to cause the light emitted by said internal light source to propagate through said light guide by internal reflection until it exits the light guide at the distal end of said laparoscope.

2. The laparoscope of claim 1 wherein the light guide is made from glass or plastic.

3. The laparoscope of claim 1 wherein the proximal end of the light guide is shaped like a lens to focus the light propagated through it at a predetermined distance in front of the objective lens or to control the size of the volume illuminated by said light or to control the angular position of the center of the illuminated volume relative to the longitudinal symmetry axis of the central core of said laparoscope.

4. The laparoscope of claim 1 comprising a metal or elastomer outer protector tube that is slipped over the light guide.

5. The laparoscope of claim 1 comprising a collar which is adapted to fit over the light guide and to be attached to the distal end of the handle section in order to keep said light guide firmly attached to said handle section.

6. The laparoscope of claim 1 comprising a transparent window that is sealed at the distal end of either the light guide or the protector tube.

7. The laparoscope of claim 1 wherein the internal light source is either LEDs or optical fibers.

8. The laparoscope of claim 3 wherein the internal light source is a ring-shaped optical fiber.

9. The laparoscope of claim 1 wherein the light guide is disposable.

10. The laparoscope of claim 4 wherein the protector tube is disposable.

11. The laparoscope of claim 5 wherein the collar is disposable.

12. The laparoscope of claim 1 comprising a centrifugal lens protector unit.

13. The laparoscope of claim 12 wherein the centrifugal lens protector unit is disposable.

14. The laparoscope of claim 1 comprising one or more working channels.
